(19) United States
(12) Patent Application Publication
(10) Pub. No.: US 2009/0093824 A1
(43) Pub. Date: Apr. 9, 2009

(54) Wound closure fasteners and device for tissue approximation and fastener application

(76) Inventors: Jafar S. Hasan, Westchester, IL (US); Wai Ngai Chin, Glenview, IL (US)

Correspondence Address: HARNESS, DICKEY & PIERCE, P.L.C., P.O. BOX 828, BLOOMFIELD HILLS, MI, 48303, US

(21) Appl. No.: 12/286,872

(22) Filed: Oct. 2, 2008

Related U.S. Application Data
(60) Provisional application No. 60/997,736, filed on Oct. 4, 2007.

(51) Int. Cl. A61B 17/10; A61B 17/12

(52) U.S. Cl. 606/139; 606/144

(57) ABSTRACT

A tissue fastening device for approximating tissue edges with successive placement of fasteners therebetween comprising a proximal portion having a handle and a trigger; a distal portion having an effector end responsive to depression of the trigger; an actuator assembly operably coupled with the trigger; approximation arms disposed in the effector end. The approximation arms are rotatably coupled to the actuator assembly; and a gear mechanism enenmeshingly engageable with the trigger. The gear mechanism is configured to rotate an arcuate needle disposed in the effector end. Method to fasten opposing tissue comprises (a) approximating two opposing tissue edges, the tissue edges comprising a dermal layer; (b) driving an H-fastener vertically through the dermal layer of each of the tissue edges using a tissue fastening device, and (c) advancing the device to an adjacent position between the tissue edges and repeating steps (a) and (b).

### CROSS-REFERENCE TO RELATED APPLICATIONS

This application claims the benefit of U.S. Provisional Application No. 60/997,736 filed on Oct. 4, 2007. The entire disclosure of the above application is incorporated herein by reference.

### FIELD

The present technology relates to methods for tissue approximation and fastening devices and fasteners for use therewith.

### BACKGROUND

In the case of skin tissue wounds, whether created through a accidental laceration, surgery or disease, healing of the wound occurs best when the layers of the skin are approximated together. In addition to proper side to side positioning (in a horizontal plane), proper wound edge positioning in the vertical plane is required to optimize healing and minimize scarring. The wound edges must be held in the optimal position for a length of time sufficient to allow for healing. When fastening wounds, the layers of the skin that must be aligned include the epidermis and the dermis. The epidermis is the superficial-most layer of the skin and it must be well aligned during wound closure to seal off the wound. However, closure of the epidermis alone may not provide sufficient strength while the wound is healing. The dermis is the deeper layer of the skin and is particularly important with regard to proper wound approximation. The dermis is the strongest layer of the skin and wound closure techniques that incorporate the dermis into the closure provide the most structural integrity.

Currently, skin staplers are often used to close wounds because they are easy to use and save substantial time in the operating room. These staplers use metal clips/staples that are inserted from the superficial skin surface. Despite efficiency and ease of use, the disadvantages of metal staples include: (1) the need to subsequently remove the staples (which can be painful for the patient) and (2) permanent marks on the surface of the skin at the staple insertion points (leading to prominent scarring which resembles a “railroad track”). To avoid these disadvantages of metal staples, surgeons may use a “layered-closure” approach in which stitches are placed in the deeper dermal layer and in the superficial epidermal layer. The deep dermal stitches are resorbable and provide tensile strength that prevents the scar from widening and becoming more prominent and allows for use of finer superficial stitches (or even glue or tape strips) as needed which leave less surface scarring. The superficial sutures may be resorbable or permanent (non-resorbable). If non-resorbable superficial sutures are used, they can be removed early (which minimizes scarring) because the deep dermal sutures remain in place to provide strength while the wound is healing. This layered closure approach is routinely used by plastic surgeons in order to minimize scarring. The key to this layered closure approach is accurate placement of resorbable sutures in the deep dermal layer.

The current method of manually placing deep dermal sutures is tedious and time consuming. In addition, manual suturing eposes healthcare workers to potential needle-stick injuries which could lead to the transmission of infectious diseases (such as hepatitis C). The ideal solution to these wound closure problems would (1) allow for efficient wound closure (especially relative to hand suturing techniques), (2) eliminate the patient discomfort associated staple removal, (3) reduce the scarring associated with traditional metal staples, and (4) reduce the risk of needlestick injuries. These goals would best be achieved by technology that places resorbable fasteners under the skin surface (in the dermal layer) in an automated fashion. Others have attempted to develop this type of resorbable deep dermal fastening system. In particular, Green et al. (U.S. Pat. Nos. 5,292,326, 5,389,102, 5,423,856, 5,489,287, and 5,573,541) designed a dermal stapler that interdigitated the wound edges in an undulating/serpentine configuration. This device then fired straight resorbable pins across the wound edge interdigitations in order to secure the edges of the wound together. This product (referred to as the AutoSuture SQS 20) was not successful for various reasons, including wound-healing problems which resulted from the undulating/interdigitating wound edge configuration. Others have attempted to create an effective dermal fastening device (U.S. Pat. Nos. 6,726,705, 7,112,214, and D0532107). However, the device of the foregoing applications have significant disadvantages including: (1) difficult/awkward method of use, (2) creation of contour irregularities in the skin (visible “dimpling” effect where the fasteners are inserted), (3) inability to bear any significant tension on the wound, (4) extrusion of the fasteners through the superficial skin surface. Some of the reasons for these shortcomings include the fastener geometry and the method of fastener insertion.

Patent Application Publication US 2007/0049969 to Peterson discloses an applicator apparatus that rotatably places non-flexible arcuate fasteners across tissue edges for approximation. Their falcate tissue penetrator “carries” a fastener and pushes the fastener through the tissue to approximate and secure the edges. The cross sectional shape of their falcate tissue penetrator is a right angle. Our drive needle is nothing like that and is instead more like a modification of a traditional suture needle. SHEET 44, FIG. 41: See parts 506a and 506b. These arms in their device serve to position the wound edges externally (i.e. superficial skin surface) which is away from the deep surface where the actual fastener insertion is occurring.

Despite these prior attempts by others, it would be desirable for surgeons and others to provide an effective and automated resorbable dermal closure system.

### SUMMARY

The present technology describes an effector end capable of driving a fastener between two tissue edges to approximate the tissues in a wound, incision and the like. The effector end can be fitted to any medical fastening device for use in tissue fastening. The effector end comprises: an arcuate needle being moveable from a resting position to an engagement position. The arcuate needle is operable to receive a fastener. The effector end also includes two or more approximator arms each being moveable from a resting position to an engagement position. A top plate and a bottom plate are disposed above the arcuate needle and the two or more approximator arms when the arcuate needle and the two or more approximator arms are in their resting state. The arcuate needle and the two or more approximator arms are disposed between the top plate and the bottom plate when approximating the tissue and inserting the needle through the tissue and releasing the fastener. At the end of the fastening steps the approximator arms and arcuate needle are in an engagement position. The arcuate needle does not penetrate the epidermis of the tissue and releases the leading retention member of the fastener in the deep dermis region of the second tissue approximated.

In a further aspect, a method for approximating or closing the edges of a wound is provided. In some embodiments, the method comprises the steps: (a) approximating a first tissue edge and a second tissue edge, the first and said second tissue edges comprising an epidermal layer, and a dermal layer; (b) driving a fastener through the lower portion of the first tissue dermal layer in an arcuate fashion across the second tissue edge. The fastener comprises a leading retention member and a trailing retention member and a longitudinal member therebetween. During fastening of the tissues the longitudinal member spans across the first and second tissue edges. The leading retention member being placed in the dermal layer of one of the tissues and the trailing retention member being placed in the dermal layer of the other tissue: and

(c) advancing the device to an adjacent position between the tissue edges and repeating steps (a) and (b).

The wound is substantially closed by advancing the device along the wound site and repeating the approximating and fastener insertion steps described above.

### DRAWINGS

The drawings described herein are for illustration purposes only and are not intended to limit the scope of the present technology in any way.

FIG. 1 is a perspective view of a illustrative tissue fastening device in accordance with the present technology.

FIG. 2 is an exploded, perspective view of an embodiment of a tissue fastening device.

FIG. 3 is a cross sectional view of the end of the wound closure device depicting the commencement of closure of the handle and trigger.

FIG. 4 is a cross sectional view of the tissue fastening device of FIG. 3 showing further closure of the handle and trigger.

FIG. 5 is a cross sectional view of the tissue fastening device of FIG. 3 showing complete closure of the handle and trigger.

FIG. 6 is a perspective cross sectional view of the effector end of tissue fastening device. This figure depicts further detail as shown in FIG. 3 depicting the commencement of closure of the handle and trigger.

FIG. 7 is a perspective cross sectional view of the effector end of tissue fastening device. This figure depicts further detail as shown in FIG. 4 depicting further closure of the handle and trigger.

FIG. 8 is a perspective cross sectional view of the effector end of tissue fastening device. This figure depicts further detail as shown in FIG. 5 showing complete closure of the handle and trigger.

FIG. 9 is an end view of end of the effector end of the tissue fastening device in its resting state.

FIG. 10 is an end view of end of the effector end of the tissue fastening device in its fully rotated state.

FIG. 11 is a perspective view of a variety of fasteners for use with the tissue fastening device.

FIG. 12 is a perspective view of an arcuate needle with a fastener leading retention member inserted into the needle's slot.

FIG. 13 is a perspective view of an arcuate needle piercing end portion showing a fastener slot and a fastener inserted into the slot for use with the tissue fastening device. A portion of the fastener has been removed for illustration purposes.

FIG. 14 is a perspective view of a hollow arcuate needle with an inserted H-fastener with a pair of approximator arms and corresponding axles.

FIG. 15 is an end view of a modified fastener inserted into a slot of an arcuate needle in accordance with the present technology.

FIG. 16 is a perspective view of the modified fastener of FIG. 15.

FIG. 17 is an end view of a modified fastener being attached to a modified piercing end of an arcuate needle.

FIG. 18 is a perspective view of a modified fastener as shown in FIG. 17.

FIG. 19 is a perspective view of an embodiment of a fastener of the present technology.

FIG. 19A is a front view of a fastener having 3 or 4 four groups of barbs aligned on the longitudinal member.

FIG. 19B is a front view of a fastener having 2 groups of barbs aligned on the longitudinal member.

FIG. 19C is a front view of a fastener having 2 groups of barbs aligned on the longitudinal member.

FIG. 20 is a top perspective view of a tissue fastening device in operation. The inset is a magnification end view of the effector end of the tissue fastening device in preparation for fastening the tissue in the tissue insertion site.

FIG. 21 is a perspective view of fastened tissue in an everted position using the tissue fastening device of the present technology.

FIG. 22 is a top end view of approximation forceps.

FIG. 23 is a side view of tip of approximation forceps.

FIG. 24 is an end view of an embodiment of the tissue fastening device fastening tissue in the tissue insertion site.

### DETAILED DESCRIPTION

The following description is merely exemplary in nature and is not intended to limit the present technology, application, or uses.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” “top” and “bottom” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology provides a tissue fastening effector end that can be used in medical fastening devices that enable automated and efficient closure of surgical incisions, wounds and approximation and attachment of surgical devices and materials to tissue. The present technology overcomes the deficiencies of the prior art by enabling approximation and fastener driving steps sequentially and by reversing the drive needle followed by tissue release in a manner that yields superior and secure tissue alignment which provides more favorable results. Moreover, wound alignment in the proper X, Y and Z axes in relation to the wound skin surface is provided using the approximation and fastening device of the present technology.

The present technology also provides a method for precisely inserting fasteners from the underside of the wound surface and into at least a portion of the dermal layers of the skin (without violating the external or superficial skin surface). The effector end generally comprises an effector end for use in a medical fastening device comprising: an arcuate needle being moveable from a resting position to an engagement position, the arcuate needle operable to receive a fastener; two or more approximator arms each being moveable from a resting position to an engagement position; and a top plate and a bottom plate disposed above the arcuate needle and the two or more approximator arms when the arcuate needle and the two or more approximator arms are in said resting state. The arcuate needle and the two or more approximator arms are disposed between the top plate and said bottom plate when in said engagement position.

For ease of illustration, the tissue approximation and fastening system comprising a tissue fastening device 10. The system and fasteners of the present technology will first be described, this will be followed by a description of the methods of using the tissue fastening device to approximate the edges of wounds and for laparoscopic use to approximate tissues to other tissues or to other substrates including prosthetic meshes, scaffolds and other materials.

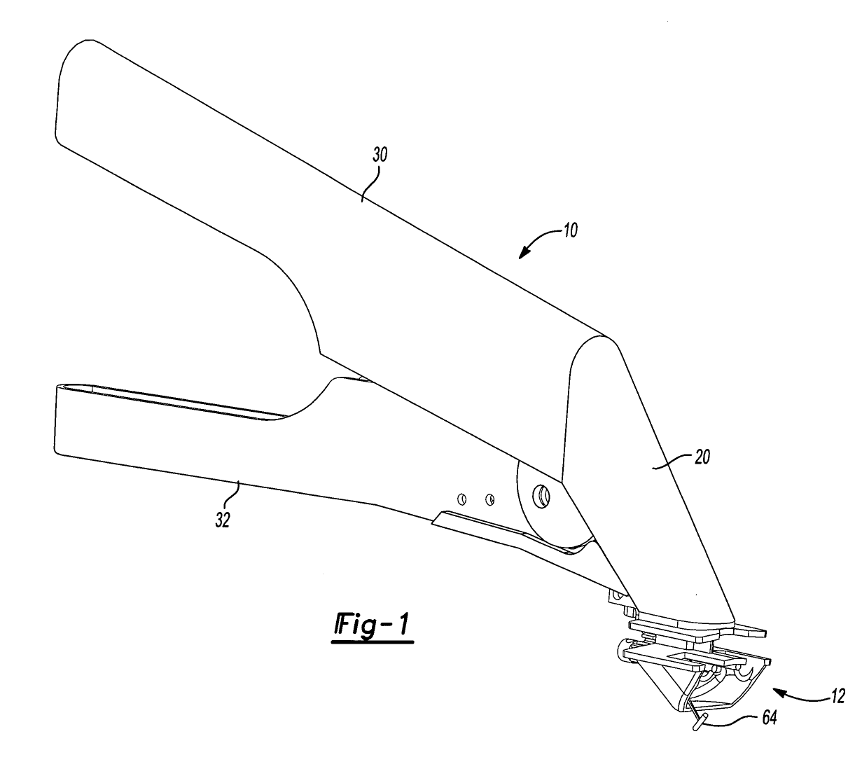

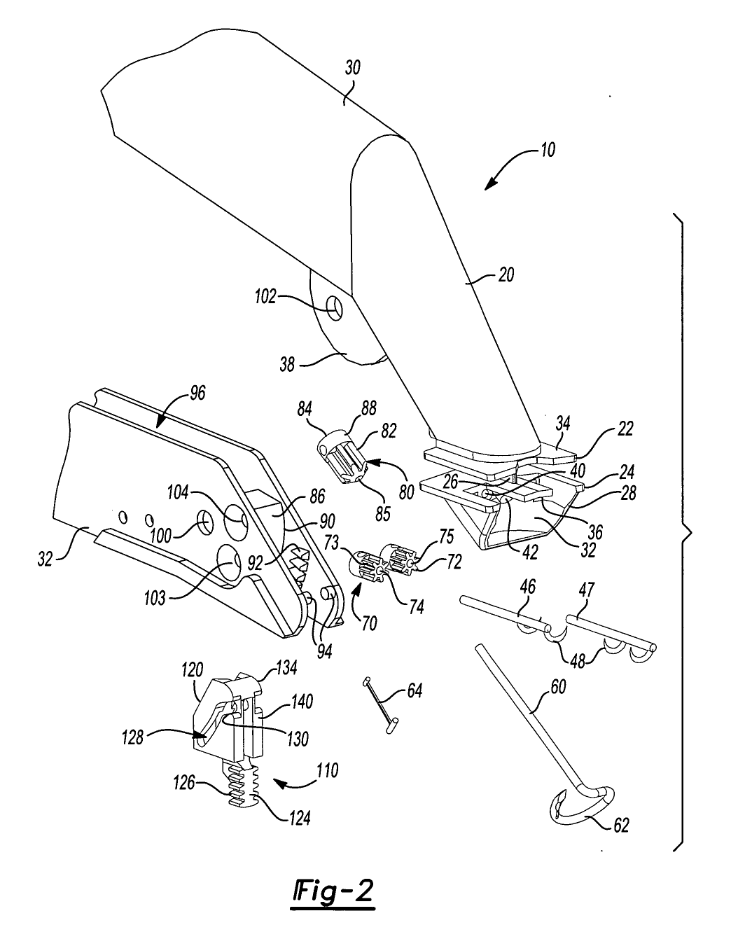

As illustrated in FIGS. 1 and 2, a tissue fastening device 10 is generally defined by a triggering end 32 and an effector end 12. The effector end can be coupled with any motorized or mechanical device capable of driving the arcuate needle axle 60 and approximator arm axle 46, 47 of the arcuate needle and the approximator arms 48 respectively. For the sake of brevity and illustration, the effector end 12 shown in FIG. 1 is accompanied with an illustrative driving mechanism consisting of gear racks, manual triggers operating on the gear racks and gears that are enmeshingly engaged with the gear racks. Other driving components can be used in the construction of the tissue fastening device 10, including for example electrical, hydraulic, pneumatic driving mechanisms that can engage and turn the approximator arm axles 46, and 47 and arcuate needle axle 60. Other methods for accomplishing the same objective are known to those of skill and are also contemplated herein. Hence, the effector end 12 can be used and practiced with other medical fastening devices which incorporate other means of axle rotation.

In some embodiments, the tissue fastening device 10 can comprise a handle 30, a device body member 20 that is part of handle 30, a trigger 32 and an effector end 12. As shown in FIG. 1, the device is in a pre-approximation state with the fastener 64 inserted into a slot of the arcuate needle. Tissue fastening device 10 can be fabricated of medically acceptable materials including metals such as, for example, stainless steel or medical grade plastic polymers. Tissue edges are held together with fasteners that are positioned under the surface of the skin. The fasteners used to approximate the edges do not need to be removed because the body can eventually resorb them. The fasteners of the present invention can be non-resorbable or resorbable. As used herein, the term “resorbable” and reabsorbable” are used interchangeably.

An exemplary embodiment of a tissue fastening device 10 of the present technology is illustrated in FIG. 2. As depicted in FIG. 2, the tissue fastening device 10 is shown in an exploded, perspective view. Handle 30 and device body 20 are connected and are generally constructed as a unitary part. Within the handle 30 is a circular member 38 that is generally attached and fixed in relation to handle 30 and device body 20. The circular member 38 contains an aperture 102 that can be coupled with an engageable rod (not shown) that is also mated with the trigger 32 through aperture 100. This enables trigger 32 to pivot about the axis of the engageable rod when the trigger 32 is depressed against the handle 30. The handle 30 and trigger 32 are displaced from one another through a spring biasing means not shown. Device body 20 couples the effector end 12 with handle 30 and provides the axis and orientation of the effector end 12 relative to the handle when the device is in use.

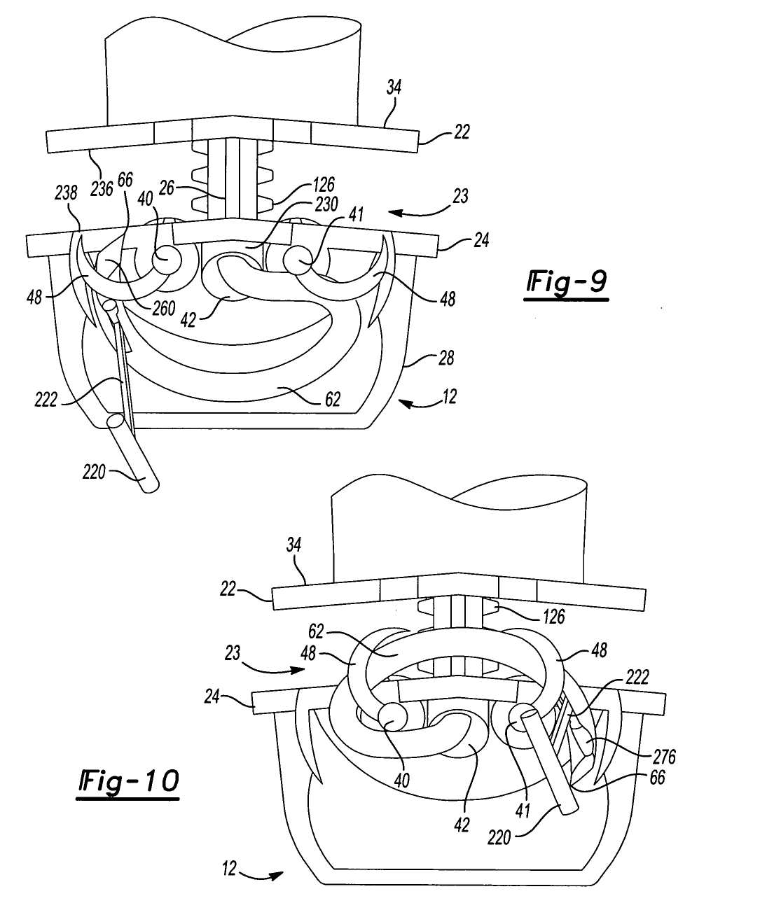

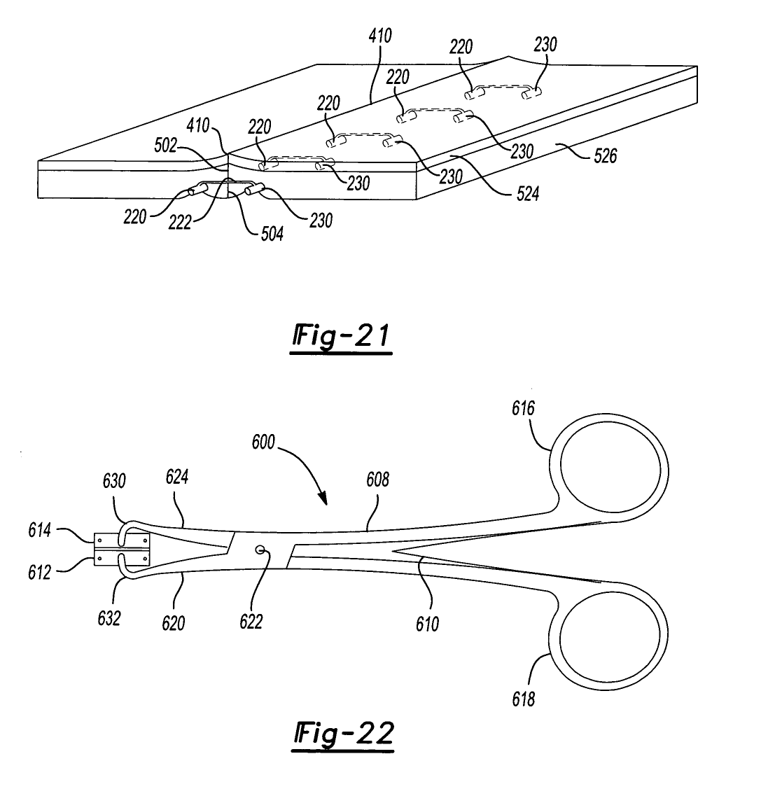

A top plate 22 and a bottom plate 24 of effector end 12 are aligned in a parallel fashion. The top and bottom plates 22 and 24 each have a central apex. The angle of each half of a horizontal plane is approximately 5°-45° best illustrated in FIG. 9. The configuration of the space created by the top plate 22 and bottom plate 24 as shown in FIGS. 9 and 10 provides additional encouragement to approximate the two tissue edges in an everted form as illustrated in FIG. 21. The top plate 22 and bottom plate 24 are separated by center partition 26. Center partition 26 prevents the approximated tissue edges from overlapping after the approximator arms 48 have grasped the tissue and approximated them together. The top and bottom plates 22 and 24 can be made from surgical metal or from hardened plastic.

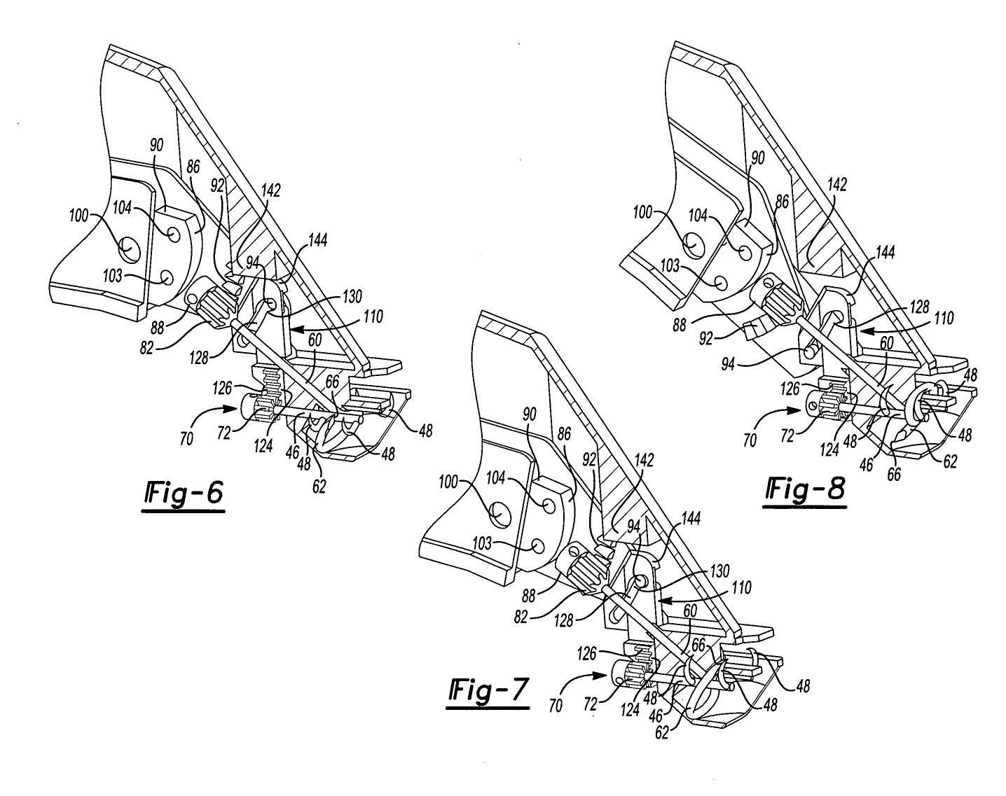

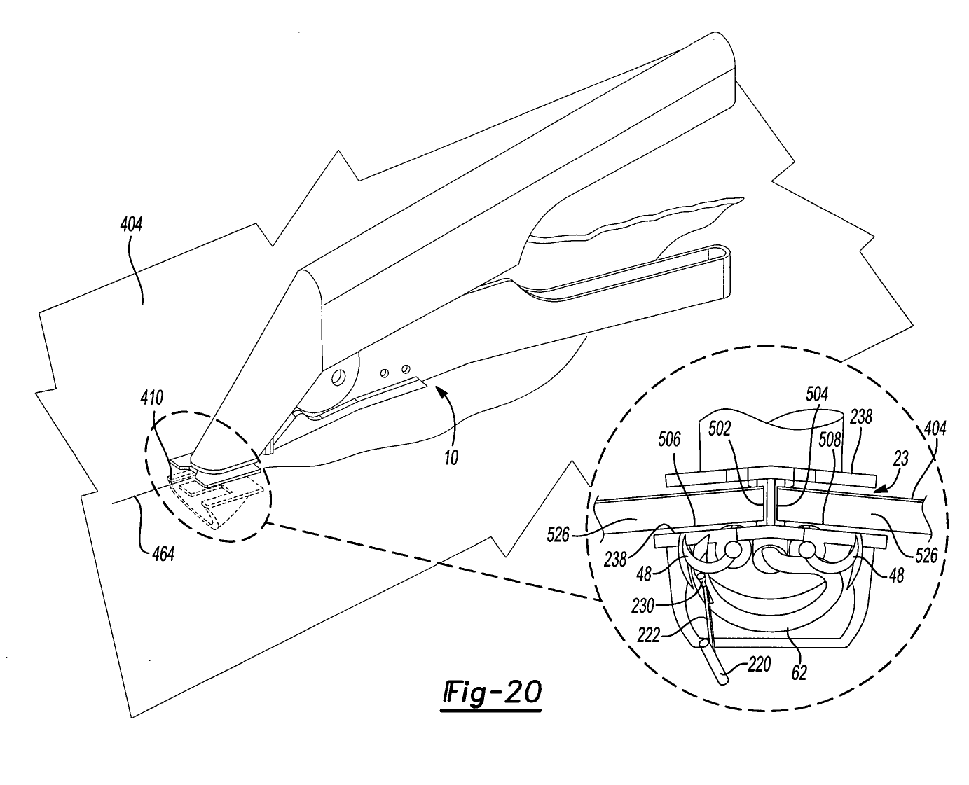

The effector end 12 includes components that provide the general fastening mechanism at the site of tissue approximation. Once the tissue fastening device 10 has been placed into position within the tissue wound or incision as illustrated in FIG. 20, the depression of the trigger 32 against the handle 30 leads to a two-stage process for dispensing a fastener between the two tissue sides 502 and 506. With reference to FIGS. 2-10 and 20, before depression of the trigger 32, actuator 110 has an actuator head surface 142 initially resting against the actuator head barrier 144 creating an engagement surface 134. At this position, the actuator channel pins 94 attached to the trigger 32 are resting in the actuator channel plateau 130 in the actuator channel 128. At this resting position shown in FIG. 9, the approximator arm gears 70 connected to approximator arm axles 47 and 46 are enmeshingly engaged with the actuator gear rack 126 on the lowest position of said actuator 110. Furthermore, the arcuate needle gear 80 also is enmeshingly engaged with the trigger gear rack 92 at the lowest position. In the resting state the approximator arms 48 and arcuate needle 62 are not in the tissue insertion site and have not commenced rotation.

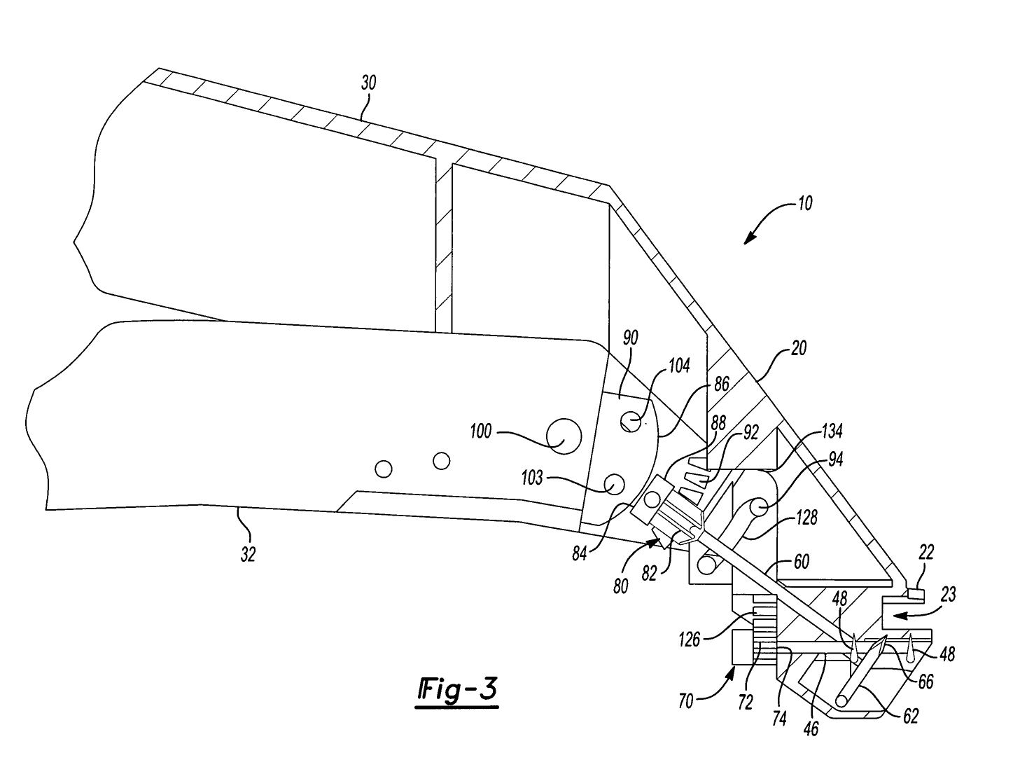

Upon compression of the trigger 32 by the operator (a surgeon, surgical assistant, nurse or other medical practitioner) moving the trigger 32 towards the handle 30 initiates the first stage of approximation. The actuator channel pins 94 biases the actuator in a downward movement as the actuator channel pins 94 exerts a force against the actuator channel plateau 130. In response of this downward movement of the actuator 110, the actuator gear rack 126 disposed on the lateral side 124 of the actuator 110 also moves in a downwards direction and rotates the approximator arm gears 70 to cause the gear rack 73 to rotate clockwise and gear rack 72 to rotate counter clockwise. Rotation of the approximator arm gears 70 concomitantly rotates the approximator arm axles 46 and 47 which are mated with their respective gears 70. The rotation of the approximator arm axles 46 and 47 translates into the rotation of their attached approximator arms 48 in a similar clockwise and counterclockwise fashion respectively. The rotation of the approximator arms 48 results in the piercing of the dermal tissue and insertion of the approximator arms into the dermal tissue thereby bringing the two tissue edges into closer contact with the center partition 26 and each other. Although there are at least two approximator arms 48 per approximator arm axle 46 and 47, the approximator arm axle 46 and 47 may comprise 2 or more approximator arms 48 per approximator arm axle. The compression of trigger 32 also induces a movement in a downward direction of the trigger gear rack 92. Since the trigger gear rack 92 is enmeshingly engaged with the arcuate needle gear 80, movement of the trigger gear rack 92 in a downward fashion causes the arcuate needle gear 80 to rotate in a clockwise fashion (when the viewer is looking from distally towards the effector end of the device). The clockwise rotation of the arcuate needle gear 80 translates to the clockwise rotation of the arcuate needle axle 60 which is mated to the rotation of the arcuate needle gear 80 as depicted in FIGS. 3-8.

Upon further compression of the trigger 32, the channel pins 94 biases the actuator in a downward movement as the channel pins 92 exert a force against the actuator channel plateau 130 until the actuator cannot travel any further in a downward direction. As a result, the approximator arms 48 connected to approximator arm axles 47 and 46 have fully rotated in their respective direction within the dermis of the tissue. This action generally results in the full approximation of the tissue contained within the tissue insertion site 23. This engagement position is illustrated in FIG. 10. Thus, the tissue edges are now fully contacting the center partition 26 near the proximal aspect of the tissue insertion site 23 and fully contacting each other at the distal portion of the tissue insertion site 23 where there is no center partition. In addition, the wound edges are slightly everted in conformance with the shape provided by the top and bottom tissue plates 22 and 24 respectively.

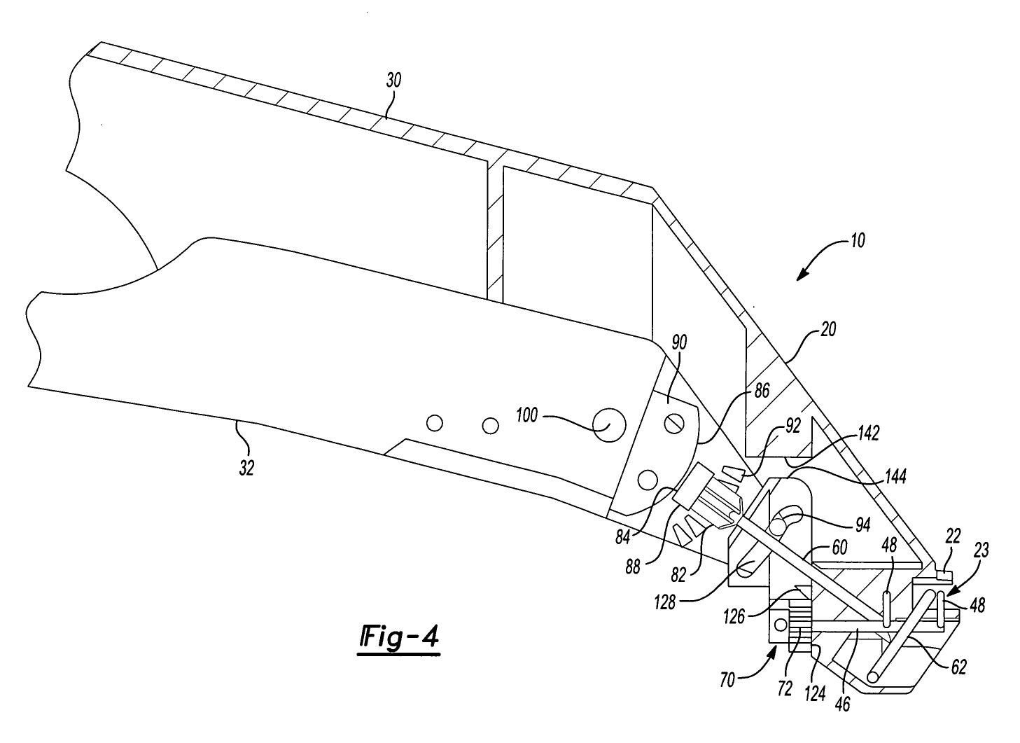

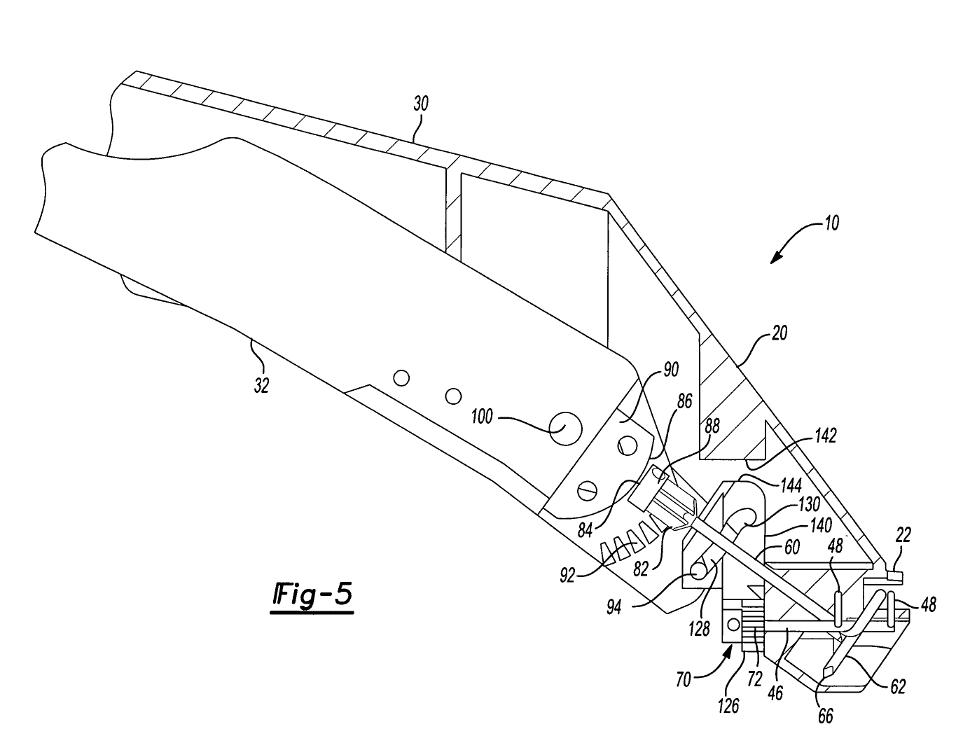

The next stage in the fastening process takes advantage of the fact that the approximator arms 48 are fully approximating the tissue edges and are being held stationary while the next stage of fastening proceeds. In order to continue the movement of the arcuate needle 62 in its arcuate path without over rotating the approximator arms 48, the trigger 32 is further compressed towards the handle 30. The trigger 32 continues to move towards the handle and advantageously compresses against the actuator ensuring that the actuator remains stationary as the actuator channel pins 94 starts to descend down the actuator channel 128. The commencement of this stage is illustrated in FIG. 7. Here the actuator channel pins 94 are at the junction of the actuator channel plateau portion 130 and the sloped portion of the actuator channel 128 and thus the further movement of the trigger 32 results in the transition of the channel pins down the actuator channel 128 as shown in FIG. 4. Since the movement of the trigger gear rack 92 continues to descend in a downwards fashion, the movement of the trigger gear rack 92 in a downward fashion causes the arcuate needle gear 80 to continue to rotate in a clockwise fashion. The clockwise rotation of the arcuate needle gear 80 translates to the rotation of the arcuate needle axle 60 and arcuate needle 62 to penetrate the deep surface of the dermis and rotate in a clockwise arcuate fashion through the dermis of both of the tissue edges 502 and 504 Advantageously, the arcuate needle 62 fails to exit through the epidermis as it proceeds to rotate through the dermal layers of both tissue sides 502 and 504. This stage can be seen in FIGS. 5 and 8. The completion of the arcuate needle 62 occurs when the trigger 32 is fully depressed against the handle 30 and the actuator channel pins has fully traversed the actuator channel 128 as shown in FIGS. 5 and 8.

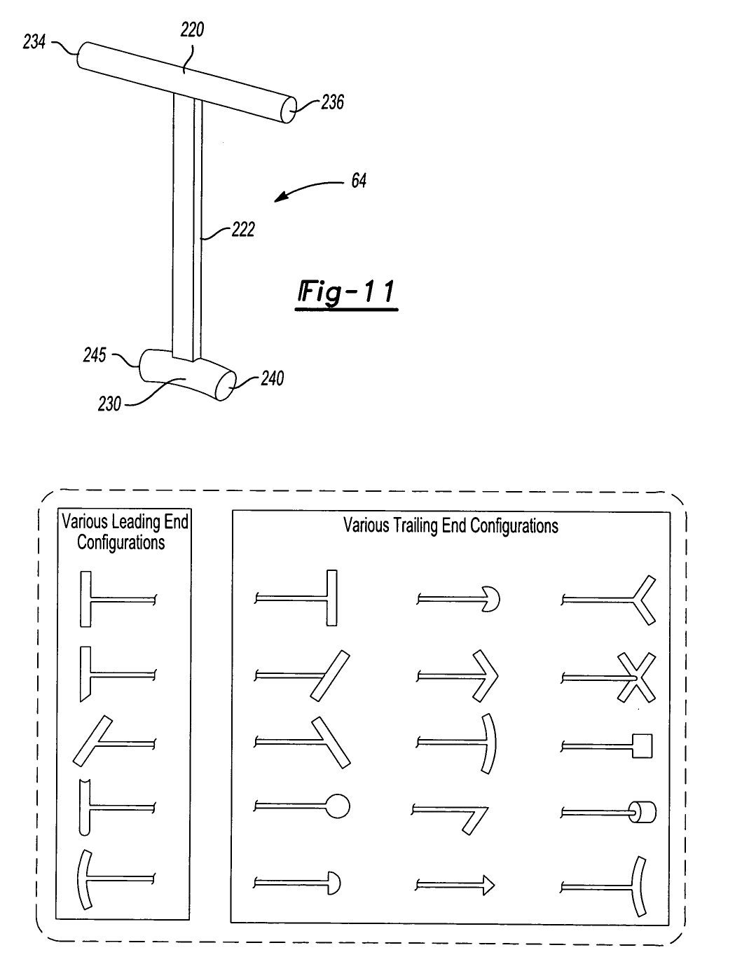

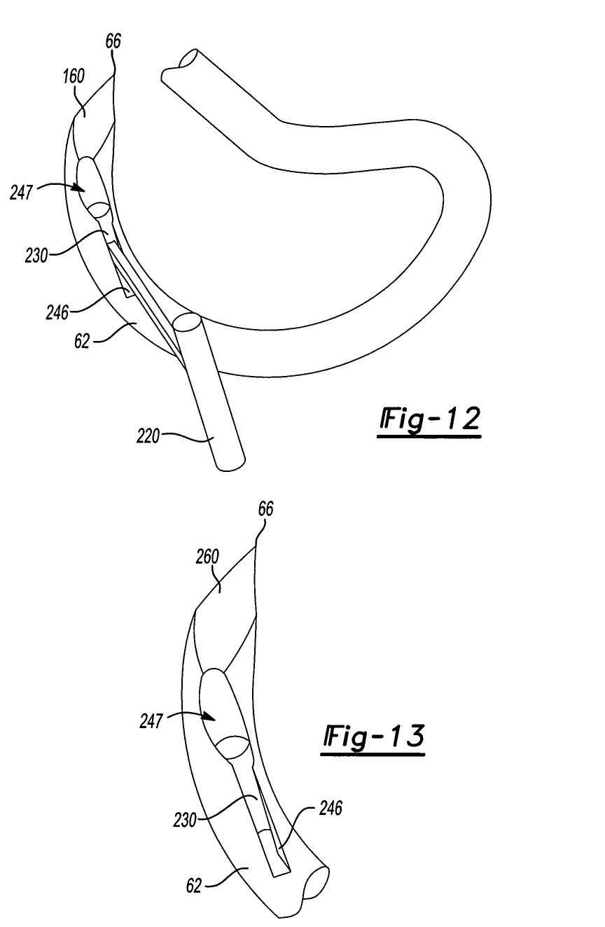

Although the tissue fastening device 10 has been described with reference to FIGS. 2-8, the tissue fastening device primary role is to fasten tissue together by the means of one or more fasteners 64. The fastener design consists of a suture-like central portion (that is flexible with regard to deviation from side but relatively inflexible along its long axis) with tissue retention elements 230 and 220 at either end. Several possible designs of the fastener 64 are depicted in FIG. 11. Generally, a fastener can have a leading retention member 230, a trailing retention member 220 and a longitudinal member 222. In some embodiments, the fastener is generally of the class commonly referred to as H-fasteners. The fasteners can be H-fasteners that are known and used in the art or they can have one or more structural modifications to at least one structural component of the H-fastener. As shown in FIG. 12, the fastener can have a trailing retention member 220 rigidly attached to a longitudinal member 222. The longitudinal member 222 is also rigidly attached to a leading retention member 230. The term leading refers to the transverse member that enters the tissue and crosses to the adjoining tissue to be fastened, and the term trailing refers to the transverse member that does not enter either of the tissues to be approximated. When the fastener is fully inserted, the trailing end abuts against the undersurface of the dermis one side of the wound, and the trailing end abuts against the undersurface of the dermis of the other side of the wound. Fastener geometries and features will be discussed below.

Returning back to FIGS. 9 and 10, a fastener 64 is shown in engagement with the arcuate needle 62. Prior to the approximation and fastening steps outlines above, the arcuate needle 62 can be fitted with a fastener 64. In some embodiments, the arcuate needle 62 has a slot 246 into which a leading retention member of a leading retention member 230 is inserted. The insertion of the fastener 64 into the arcuate needle 62 can be manual or can be automatic, for example through an attached cassette or cartridge of fasteners, mechanized for loading one fastener into the arcuate needle per trigger depression. Once the fastener leading retention member 230 is loaded into the arcuate needle fastener slot 246 and the tissue fastening device is positioned appropriately within the tissue opening, the depression of the trigger 32 will result in the approximation of the tissue edges within the tissue insertion site 23. The approximator arms 48 can rotate into the two tissues through the deep dermis region and approximate the tissues against the center partition 26 near the proximal portion of the tissue insertion site 23 and against each other near the distal portion of the tissue insertion site 23 where there is no center partition. Once the approximation of the tissue edges is complete, the next stage in passing the arcuate needle 62 through a first tissue and through into a second tissue bringing with the arcuate needle 62 an attached fastener via the leading retention member 230. Once the fastener loaded arcuate needle piercing end 66 and arcuate needle fastener slot 246 has traversed past the deep surface of the dermis of the approximated tissue to be fastened, the leading retention member 230 exits the arcuate needle fastener slot 246 and resumes an orthogonal orientation in relation to the longitudinal member 222 upon retraction of the arcuate needle 62, from the approximated tissue.

Once the fastener has been inserted, the arcuate needle 62 needs to be de-rotated back out of the two tissue edges 502 and 504. The arcuate needle 62 is retracted along the same path as was used for inserting the leading retention member 230. If the approximator arms 48 were to release the wound edges prior to de-rotation of the drive needle, the smooth path that the drive needle created on its way in would be altered. This alteration in the tissue configuration would make it difficult for the arcuate needle 62 to rotate back out of the wound. In other words, the arcuate needle 62 would bind up on the surrounding tissue. To prevent this problem, the approximator arms 48 keep the wound edges firmly approximated until after the fastener insertion needle has rotated out of the wound. This sequence is accomplished by reversing the triggering mechanism described above. Essentially, as the trigger 32 is being released and commences to move away from the handle 30, the movement of the trigger away from the handle causes the trigger gear rack 92 to start moving up and this motion induces the enmeshingly engaged arcuate gear rack 82 to rotate the arcuate needle gear 80 and cause the arcuate needle to retract to its resting position. This rotation of the arcuate needle gear 80 then rotates the arcuate needle axle and causes the arcuate needle 62 to rotate in an arcuate fashion, exactly the opposite form the way in which the arcuate needle 62 was originally inserted. As the trigger 32 continues to move to its original resting position, the actuator channel pins 94 begin to move up along the actuator channel 128. When the trigger 32 is approximately released half-way, the actuator channel pins 94 begin biasing the actuator to move upwards towards the actuator head barrier 144. Once the actuator 110 starts to move upwards, the actuator gear racks 126 also begin to move in an upward direction. When the actuator gear rack 126 disposed on the lateral side 124 of the actuator 110 move in an upwards direction, the enmeshingly engaged approximator arm gears 70 connected to approximator arm axles 46 and 47 rotate away from an engaged position towards a resting position. Rotation of the approximator arm gears 70 concomitantly rotates the approximator arm axles 46 and 47 which are mated with their respective gears 70. The rotation of the approximator arm axles 46 and 47 translates into the rotation of their attached approximator arms 48 in a similar fashion The rotation of the approximator arms 48 on approximator arm axle 46 and the opposite rotation of the approximator arms 48 on the approximator arm axle 47, results in the retraction of the approximator arms 48 from the dermal tissue back towards its resting position as shown in FIG. 9. The tissue fastening device can then be moved about 3 mm to about 30 mm along the open wound, incision or tissue opening to recommence the placement of the next fastener.

Hence, the present device advantageously retracts the arcuate needle 62 first and approximator arms 48 second in a coordinated fashion that minimizes the displacement of the inserted fastener and prevents binding of the arcuate needle 62 on the surrounding tissue upon retraction of the arcuate needle 62 and approximator arms 48.

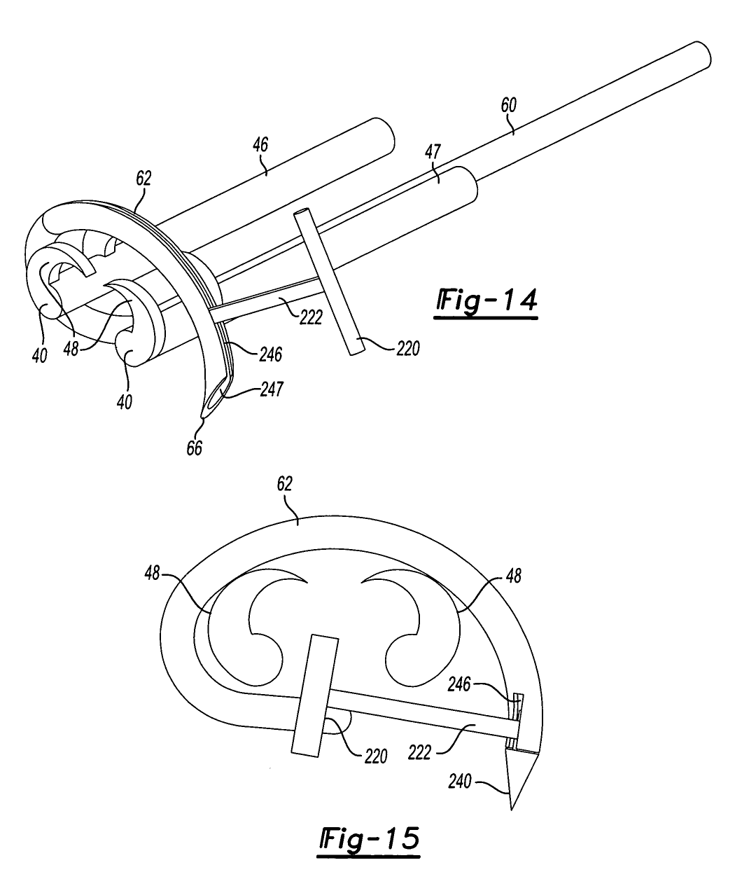

In some embodiments, the fastener 64 can be modified to adjust to the form of the arcuate needle 62 and arcuate needle fastener slot 246. With reference to FIG. 11, a fastener having a slightly curved leading retention member 230 can fit into and conform to the shape of the arcuate needle 62. The length of the leading retention member 230 can vary depending on the length and size of the arcuate needle fastener slot 246. As shown in FIGS. 12 and 13, a fastener having a short length leading retention member 230 can advantageously fit into an arcuate needle fastener slot having a longitudinal member 222 that is capable of being partially included into the slot for ease of passage through the dermis tissue. Typically, the diameter of the arcuate needle fastener slot 246 is slightly larger than the diameter of the leading retention member 230. In some embodiments, the arcuate needle fastener slot 246 can be placed in the arcuate needle proximate to the piercing end 66. The arcuate needle fastener slot 246 can also be positioned on the arcuate needle 62 near or proximate to the stack of fasteners to be inserted into the arcuate needle fastener slot 246. As shown in FIGS. 13 and 14, the piercing tip can comprise a surgically manufactured needle tip having one or more sides or facets. Illustratively shown in FIGS. 13 and 14, the arcuate needle is shown with piercing tip 66 having 2 facets. In FIG. 13, the pierce tip 66 can have 2 or 3 facets. In some embodiments, the piercing end 66 can be any surgically useful tip that can both cut and pierce at the same time.

In some embodiments, a variation on the arcuate needle 62 is illustrated in FIG. 14. A fastener 64 is slidably positionable in the arcuate needle fastener slot 246. In this embodiment, the arcuate needle fastener slot opening 247 is located on the piercing face 260 (or just proximal to the piercing face 260), which accepts the leading retention member 230 into the arcuate needle fastener slot 246 and is either entirely contained within the arcuate needle fastener slot 246 or at least the leading retention member 230 and its junction with the longitudinal member 222 is contained within the arcuate needle fastener slot 246. When the arcuate needle 62 has penetrated both tissues to be approximated and has arrived at its terminal position in the dermis tissue as shown in FIG. 14, a push rod member (not shown) can be extended along the lumen of the arcuate needle fastener slot 246 and extends the leading retention member 230 from the arcuate needle fastener slot 246 at the fastening position in the tissue.

In some embodiments, a tissue fastening device 10 is illustrated in FIG. 24. A pair of approximator arms 48 approximate the tissue placed in the tissue insertion site 23. The arcuate needle 62 fitted with a fastener 64 with a leading retention member 230 inserted into an arcuate needle (not shown) is rotated counter clockwise through the dermis 526 of the two tissue edges to be fastened. After the arcuate needle 62 has reached the terminal position as illustrated in FIG. 24, a pusher arm 706 is rotated through the interior of the arcuate needle 62 and urges the leading retention member 230 to pass through the arcuate needle until the leading retention member 230 exits the arcuate needle fastener slot 246 and into the dermis 526 of the second tissue edge to be fastened. The arcuate needle then retracts clockwise back to its resting position, leaving the fastener in place in order to fasten the tissue edges together.

As illustrated in FIGS. 11, 15-19, fasteners generally comprise a leading retention member end 230 rigidly attached to a longitudinal member 222. The longitudinal member 222 is also rigidly attached to a trailing retention member 220. The dimensions of the leading and trailing retention members 230 and 220 respectively can vary with the intended arcuate needle design and the mode of placement of the fastener 64 in or with the arcuate needle 62. Each of the leading and trailing retention members 230 and 220 have a first end 245 and 234 respectively and second ends 240 and 236 respectively. The longitudinal member 222 cross-section may be circular, square, triangular or any geometric shape.

The longitudinal member 222 can also include one or more barbs along the surface as shown in FIGS. 19 A-C. The barbs can be positioned adjacent the leading retention member 230 end rather than the trailing retention member 220. They can be randomly positioned near the leading retention member 230 end or they can be positioned in rows as shown in FIG. 19A-19C. One advantage imparted to fasteners 64 having such barbs 810 is that the fastener 64 is less likely to be displaced after the leading retention member has exited the arcuate needle 62 and the arcuate needle 62 has begun to be retracted along the same path as was used to insert the fastener 64. In such cases, the barbs 810 ensure that the fastener 64 will not move along with the arcuate needle 62 as the arcuate needle begins to rotate back out of the wound.

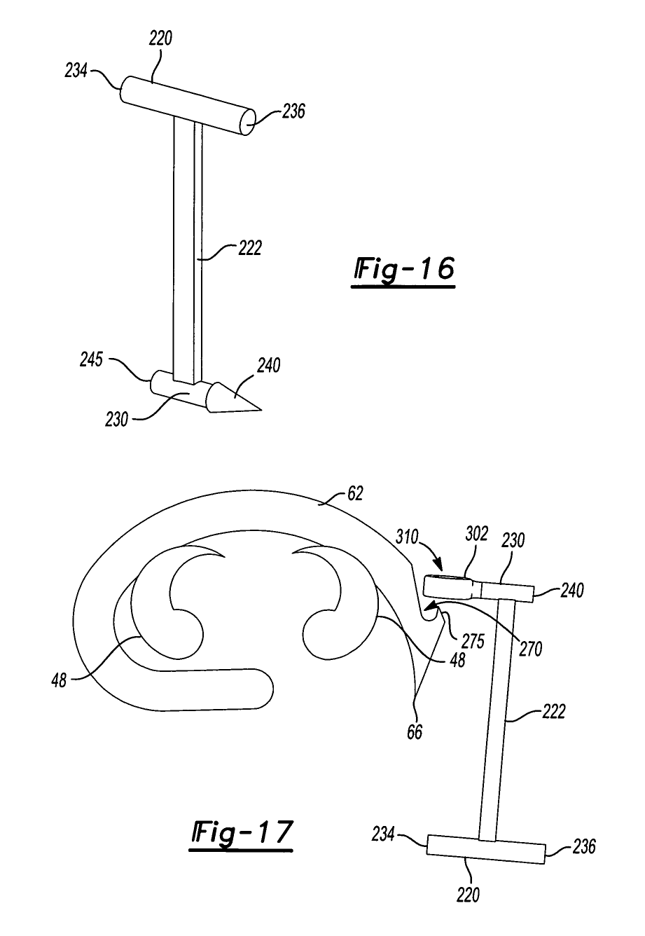

In some embodiments, the leading and trailing retention members 230 and 220 respectively can be shaped to be the same or be different. In some embodiments, the second end of the leading retention member 240 can include a cone (as shown in FIG. 16) or other pointed geometry, for example a triangle, a needle, a spire and the like, to assist in the piercing of the arcuate needle 62 equipped with a pointed leading retention member 240. A leading retention member having such a geometry, is particularly advantageous, when the arcuate needle 62 is rotated in a clockwise manner and is configured to pierce the two tissue edges to be approximated.

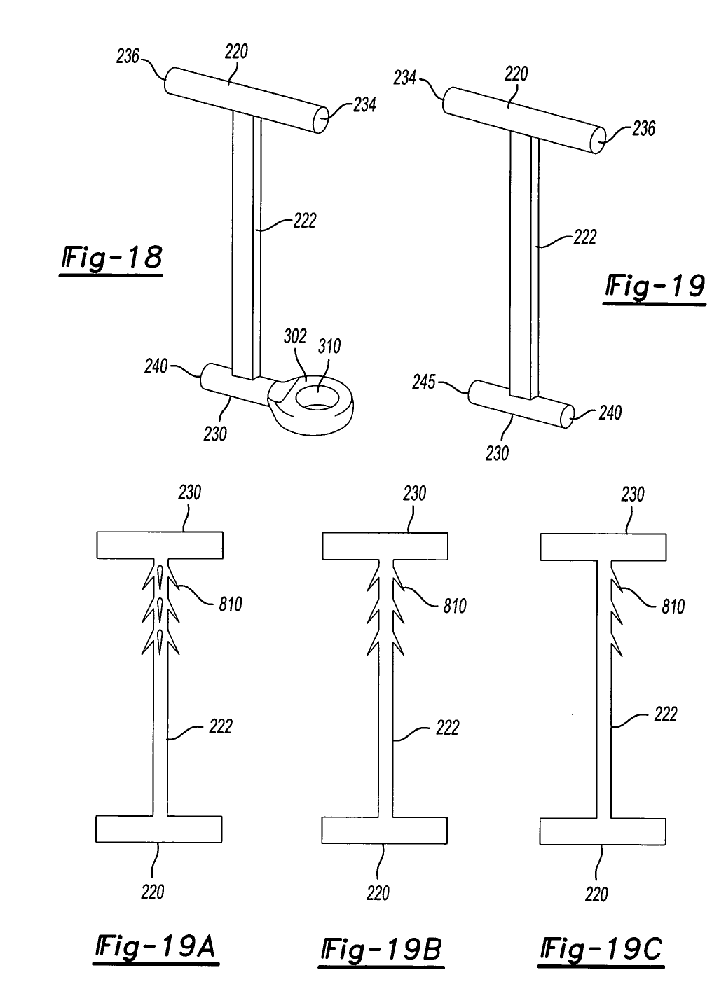

In some embodiments, the arcuate needle 62 shown in FIG. 17 has a cleat 270 defining a cleat space 270. The general resemblance of cleat 270 is similar to a barb on a hook 275. Arcuate needle 62 as shown in FIG. 17 rotates in a clockwise manner as described previously, and when the arcuate needle has reached the terminal position of its rotation as shown in FIG. 17, the cleat 270 is contacted with a fastener having a capture ring 302 as illustrated in FIG. 18. The capture ring 302 connected to the leading retention member 230 has a capture ring aperture 310 that can mate with cleat 270 on the arcuate needle 62. When the arcuate needle 62 is retracted, the arcuate needle couples with the fastener 64 through the capture ring 302 and retrieves the fastener 64 through the arcuate path created by the arcuate needle 62 and decouples from the capture ring 302 when it has reached its initial fastening position as shown in FIG. 9 leaving the trailing retention member 220 on the initial fastener placement side and places the leading retention member 230 on the other tissue side to be fastened.

In some embodiments, the fasteners of the present technology can be non-resorbable, or absorbable. In some embodiments, the fastener 64 includes a generally bioabsorbable polymer selected to maintain effective retention strength for a period of at least 5 to 21 days within the body, and optimally at least 14 days before eventually being fully absorbed within the human body. General criteria for the materials to be selected in the manufacturing of the fasteners of the present technology can include biocompatibility and fastener deformability. The fasteners 64 of the present technology are easily deformable when the leading and transverse members are displaced along a vertical axis (i.e. bending of the longitudinal member to either side of its long axis). However, the fasteners 64 are very resistant to any stretching or elastic deformation when the leading and transverse members are displaced along a horizontal axis (i.e. high tensile strength along the long axis of the longitudinal member). Such selection of H-fasteners described herein offer several advantages over the preexisting field, for example, the curved solid fasteners of the preexisting field rely on resistance to tissue stretching when the curved fasteners are placed either horizontally or vertically in relation to the surface of the skin. Such resistance is mediated by the bending strength of the fastener. In contrast, the fasteners 64 used in the present technology are very flexible (in a direction lateral to the long axis of the longitudinal member but not along the long axis of the longitudinal member), have a lower profile, and can accommodate any arcuate needle design. The fasteners 64 are not bulky and have a lower profile and therefore require less force to extend them across the tissue edges. In addition, the lower profile of the longitudinal member 222 of the fasteners 64 translates to less material being present in the wound edge interface, which will result in less inflammatory response. The fasteners 64 of the present technology rely on tensile strength to keep the tissue edges approximated and fastened even days and weeks after placement. The fasteners of the present technology offer the convenience of staples but function more like suture material than staples or solid arcuate fasteners of the prior art.

Examples of the non-absorbable biocompatible materials which can be used in the manufacture of the fasteners 64 include polypropylene, nylon, polyethylene, polyester polyolefin and the like and equivalents thereof. Exemplary absorbable and resorbable biocompatible polymeric materials that can be used to manufacture the H-fasteners of the present technology can include polydioxanone, polygalactic acid, polylactic acid, polycaprolactone, lactide/glycolide copolymer, a poly(dl-lactide), a poly(l-lactide), a polyglycolide, a poly(dioxanone), a poly(glycolide-co-trimethylene carbonate), a poly(l-lactide-co-glycolide), a poly(dl-lactide-co-glycolide), a poly(l-lactide-co-di-lactide), a poly(glycolide-co-trimethylene carbonate-co-dioxanone), collagen, and elastin, either individually, in blends or as copolymers as well as equivalents thereof. The polymers can be mixed with tissue growth enhancing materials such as calcium ceramics; such as calcium phosphates, calcium sulfates, calcium phosphate apatites; antibiotics, e.g. aminoglycosides, ansamycins, carbacephem, carbapenems, cephalosporins, glycopeptides, macrolides, monobactams, penicillins, polypeptides, quinolones, sulfonamides, and tetracyclines; anti-inflammatory agents, e.g. non-steroidal anti-inflammatory drugs, naproxen, ibuprofen, aspirin; collagen and tissue growth factors involved in wound healing, for example, TGF-β, PDGF, VEGF, EGF, fibronectin and mixtures thereof and the like. In some embodiments, the H-fasteners of the present technology can be manufactured from other conventional types of biocompatible materials including metals such as stainless steel, spring steel and nickel-titanium alloys (e.g., Nitinol), ceramics, composites, and the like and equivalents thereof.

Methods of Using the Tissue Approximation System

The tissue fastening device 10 can be utilized by surgeons, physicians, nurses, surgical assistants and other medical personnel to accomplish a variety of skin closures including intentional, surgical incisions as well as accidental lacerations. Generally, a first step in effectuating wound closure with tissue fastening device 10 is to position the effector end 12 in the wound as illustrated in FIG. 20. Tissue fastening device 10 should be in a pre-firing orientation, for example pre-fastener deployment configuration as illustrated in FIGS. 1 and 9. Once the effector end 12 has been inserted into the wound and having the wound edges placed into the tissue insertion site 23, optionally with the aid of approximation forceps 600, the surgeon can depress the trigger 32 to cause the tissue fastening device 10 to internally approximate the first and second tissue edges 502 and 504 and fire a fastener across these approximated wound edges. As shown in FIG. 20, the tissue fastening device 10 need only be inserted into the wound or incision only as far as to align the two tissue edges between the top and bottom plates. Pulling the trigger 32 automatically places the wound edges in a favorable configuration (in 3 dimensions) then coordinates this alignment with the fastener insertion. The standard placement and repeatable fastener placement in a precisely aligned manner results in increased ease of use, increased efficiency and also more favorable clinical outcomes (i.e. less chance of scarring) due to superior wound alignment. In addition, the user of the device is not exposed to the risk of needle stick injuries as is the case with manual suture placement.

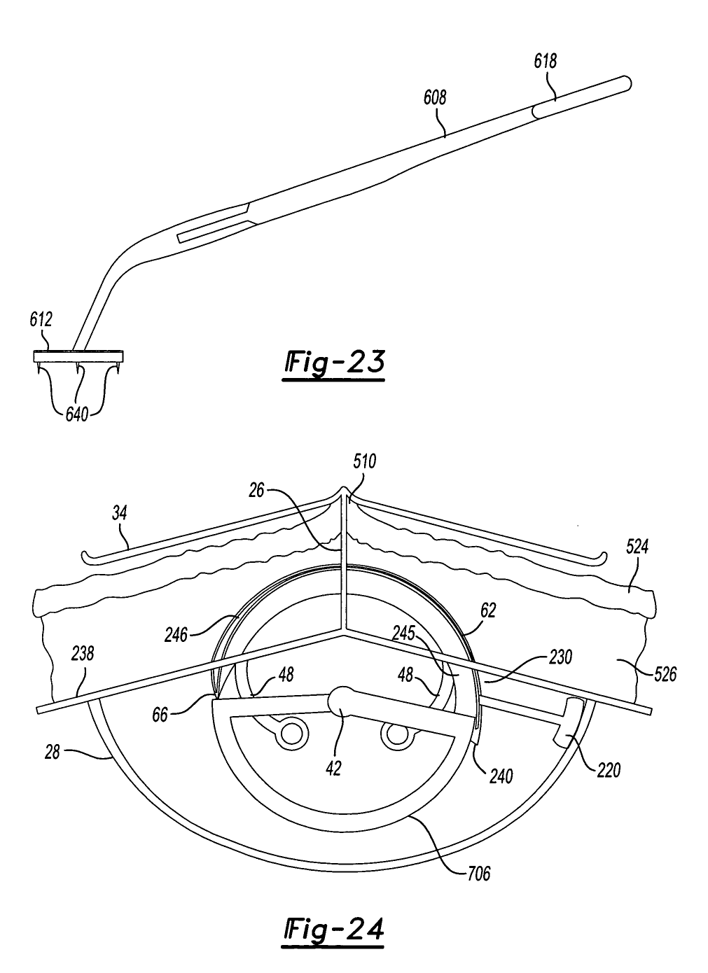

As illustrated in FIGS. 20-23, while positioning the device in a wound to be closed or approximated, an approximation forceps 600 may be used to help initially position the wound edges into the tissue insertion site 23 of the tissue fastening device 10. The approximation forceps 600 will not always be needed for application of the device and will most often be useful when there is significant gapping of the wound edges to be closed (e.g. when large amounts of tissue have been excised.) The approximation forceps 600 can be an adaptation of any type of surgical forceps as illustratively shown in FIGS. 22-23. The approximation forceps 600 can include a forceps design having convenient features, including a pair of finger handles 616 and 618 operatively connected to a forceps body 608. The finger handles 616 and 618 can be biased against each other with a bias means that can include a spring mechanism using two metal plates as shown in FIG. 22, or they may be biased using a coiled spring. Surgical forceps used for grasping can be modified by welding or otherwise adhering a base plate 612 and 614 onto a forceps connector 630 and 632. Each arm of the forceps 620 and 624 terminate in a pair of base plate connectors 630 and 632 which are adhered to base plates 612 and 614 having one or more tines 640. The tines 640 can be measure about 1 to about 10 mm long, 0.1-3 mm in diameter at the base plate and can be sharpened to a point. The tines 640 can be generally made of surgical steel or plastic. The surgical forceps should be made to withstand standard hospital sterilization procedures. The tines 640 are used to engage the superficial skin surface 404, allow the tissue edges 502 and 504 to be moved closer together. In order to facilitate engagement of the skin surface, the tines 640 may be angled inward towards the midline (ideally 5 to 80 degrees inwards relative to a position perpendicular to the base plates). The tapered/sharpened tip and angle of the tines is designed to engage the skin without causing tears (lacerations or abrasions) that lead to additional scarring. These forceps can be especially useful when the wound edges are gaping apart.

As illustrated in FIGS. 20 and 21, the tissue fastening device 10 can be inserted into a wound or surgical incision and used to approximate and fasten tissue edges using embedded fasteners 464 and form a tissue approximation line 410. A fastener can be used to approximate tissue edges every 3 to 20 mm along the tissue approximation line. As shown in FIG. 21, the fastening methods described herein with the tissue fastening device described above result in slightly everted tissue edges to a general closure configuration which is known to be clinically beneficial. The fasteners comprising the leading and trailing retention members 230 and 220 respectively and longitudinal member 222, become positioned in a generally horizontal manner after the tissue edges 502 and 504 exerts some force against the fasteners. This horizontal (and relatively linear) fastener position allows the fasteners to hold the wound edges together securely as a result of the high tensile strength along the long axis of the longitudinal member 222. In some embodiments, the tissue to be approximated and fastened can include 2 patient tissues, one patient tissue and one artificial tissue, or two artificial tissues. The artificial tissue can include materials such as prosthetic mesh or other substrates and materials anchored to tissue during operations such as laparoscopic hernia repair (rather than using the fasteners 64 to close surgical wounds). Other synthetic tissues can include artificial tendon, artificial cartilage, artificial ligaments, and biodegradable membranes.

In some embodiments, a laparoscopic device is provided wherein fasteners 64 can be used to anchor materials such as prosthetic mesh or other substrates and materials to tissue during operations such as laparoscopic hernia repair (rather than using the fasteners 64 to close surgical wounds). The fastening device 10 disclosed in the present technology can also be used in any type of anchoring/fastening application during laparoscopic surgery (i.e. not just for fastening mesh to tissue). This technology could also be used in any situation where access to the fastening site is difficult (i.e. not just during laparoscopic operations).

In some embodiments in accordance with the present technology, a laparoscopic fastening device is provided that comprises an elongate tubular member having a distal potion and a proximal portion. A distal portion of the laparoscopic device can be inserted into a wound or laparoscopic port site wherein the distal portion has a drive/fastener means as illustrated for the tissue fastening device 10. The laparoscopic tissue approximation device has a proximal portion that can be connected to an actuator comprising a triggering mechanism as described above for the tissue approximation system. The triggering mechanism can be operably coupled to the drive/fastener mechanism; and the fastening of one or more fasteners. The triggering means upon actuation engages said drive/fastener mechanism; and the drive/fastener means can comprise a rotatable slotted arcuate needle 62 and one or more approximator arms 48 operable rotationally as described above for the tissue fastening device 10.

Various laparoscopic fastening methods exist, including variations of the traditional H-fastener such as the I-Clip™ Tissue Fixation System by Autosuture (Covidien Ltd., Mansfield, Mass., USA) The current methods of H-fastener insertion involve linear penetration of the tissue followed by advancement of the H-fastener. This linear insertion is sub-optimal because of the following reasons: 1. The linear insertion is performed in a “blind fashion” (i.e. the needle is inserted linearly into the adjacent tissues, creating the potential for damage of critical structures). 2. The linear fastener insertion is performed at the tip of the device, in line with the longitudinal axis of the body of the device. This configuration makes it difficult to position the device for fastener insertion in certain situations. 3. The leading end of the fastener is inserted in a “buried” fashion in the adjacent/underlying tissue. That method makes fastener insertion (i.e. secure anchoring) more difficult.

In some embodiments, the method of rotatably inserting the fasteners via an arcuate needle 62 solves the problems described herein in the following ways. Arcuate insertion of the fastener results in an almost tangential “bite” of the underlying tissue which provides the needed support without requiring deep penetration. The result is a diminished potential for damage of the underlying tissue structures. The arcuate insertion of the fastener allows the effector end 12 of the tissue fastening device 10 to be positioned against the site where fastening is required, which makes fastener application easier. In some embodiments, where laparoscopic use of a tissue fastener is required, both the leading and trailing ends of the fasteners lie on the same side of the mesh while the longitudinal portion of the fastener crosses the mesh to lie in an essentially horizontal course in the adjacent tissue. This configuration results in improved fastener holding strength and reliable insertion because the leading end of the fastener does not lie in a “buried” position.

What is claimed is:

1. An effector end for use in a medical fastening device comprising:
an arcuate needle being moveable from a resting position to an engagement position, said arcuate needle operable to receive a fastener;
two or more approximator arms each being moveable from a resting position to an engagement position; and
a top plate and a bottom plate disposed above said arcuate needle and said two or more approximator arms when said arcuate needle and said two or more approximator arms are in said resting state;
wherein said arcuate needle and said two or more approximator arms are disposed between said top plate and said bottom plate when in said engagement position.

2. The effector end of claim 1, wherein said arcuate needle comprises a piercing tip having at least one facet adapted to pierce a tissue disposed in said tissue insertion site.

3. The effector end of claim 2, wherein said arcuate needle includes a fastener receiving portion including a slot or hook proximate to said piercing tip

4. The effector end of claim 1, wherein said arcuate needle comprises a rotatable axle coupled to said driving component.

5. The effector end of claim 1, wherein said arcuate needle comprises a hollow lumen disposed through at least 50% of said arcuate needle.

6. The effector end of claim 1, wherein said two or more approximator arms are arcuate.

7. The effector end of claim 6, wherein said two or more approximator arms are attached to a pair approximator arm axles.

8. The effector arm of claim 7, wherein said approximator arm axles are rotatably coupled to said driving component.

9. The effector end of claim 1, wherein said top plate and said bottom plate are oriented in parallel fashion, said top plate and said bottom plate sloping down from a center apex.

10. A tissue fastening device comprising:
an effector end having:
an arcuate needle being moveable from a resting position to an engagement position, said arcuate needle operable to receive a fastener;
two or more approximator arms each being moveable from a resting position to an engagement position;
a top plate and a bottom plate disposed above said arcuate needle and said two or more approximator arms when said arcuate needle and said two or more approximator arms are in said resting state;
a driving component operably coupled to said arcuate needle and said two or more approximator arms; and
a handle operably connected to said effector end.

11. The tissue fastening device of claim 10, wherein said arcuate needle is coupled to a rotatable arcuate needle axle, said arcuate needle axle coupled to said driving component

12. The tissue fastening device of claim 10, wherein said first approximator arm and said second approximator arm are attached to a first approximator arm axle and a second approximator arm axle respectively.

13. The tissue fastening device of claim 10, wherein the driving component comprises a trigger having a trigger gear rack and an actuator pin, an actuator slidably engageable with said actuator pin, said actuator having a gear rack, a first gear member enmeshingly engageable with said trigger gear rack and coupled to an arcuate needle axle and a first approximator arm gear and a second approximator arm gear, said first approximator arm gear and said second approximator arm gear enmeshingly engageable with said actuator gear rack.

14. The tissue fastening device of claim 10 operably inserted into a laparoscopic device.

15. A method for fastening two opposing tissues in a patient, said method comprising:
(a) approximating a first tissue edge and a second tissue edge, said first and said second tissue edges comprising an epidermal layer, and a dermal layer;
(b) driving a fastener through the lower portion of said first tissue dermal layer in an arcuate fashion across said second tissue edge, said fastener comprises a leading retention member and a trailing retention member and a longitudinal member therebetween, wherein said longitudinal member spanning across said first and second tissue edges, said leading member being placed in said dermal layer of one of said tissues and said trailing member being placed in said dermal layer the other of said tissues: and
(c) advancing said device to an adjacent position between the tissue edges and repeating steps (a) and (b).

16. The method of claim 15, wherein said driving a fastener through a dermal layer comprises:
providing a medical fastening device comprising an effector end, said effector end comprising an arcuate needle being moveable from a resting position to an engagement position, said arcuate needle operable to receive a fastener;
two or more approximator arms each being moveable from a resting position to an engagement position;
a driving component operably coupled to said arcuate needle and said two or more approximator arms; and
a top plate and a bottom plate disposed above said arcuate needle and said two or more approximator arms when said arcuate needle and said two or more approximator arms are in said resting state;
inserting said medical fastening device between two tissues, said tissues each having a tissue edge;
inserting said tissues between said top plate and said bottom plate;
inserting one or more fasteners in said medical fastening device; and
applying said one or more fasteners across said tissue edges,
wherein said arcuate needle rotates through the bottom of said tissues and traverses across said tissue edges and terminates said rotation at said engagement position.

17. The method of claim 16, wherein said method further comprises:
approximating said tissue edges using an approximation forceps to assist in properly positioning the tissue edges in the tissue recess of said medical fastening device

18. The method according to claim 16, wherein the fasteners are resorbable.

19. The method according to claim 18, wherein the fasteners further comprise a plurality of barbs.

20. The method of claim 15, wherein one of said tissues is an artificial tissue comprising a mesh, an artificial tendon, an artificial cartilage, an artificial ligament, and a biodegradable membrane.
